(19) United States
(12) Patent Application Publication
(10) Pub. No.: US 2022/0218368 A1
(43) Pub. Date: Jul. 14, 2022

(54) THROMBUS REMOVAL DEVICE

(71) Applicant: Taiwan Biomaterial Co., Ltd., Zhubei City, Hsinchu County (TW)

(72) Inventors: Chun-Jen LIAO, Zhubei City (TW); Wen-Hsiang CHANG, Zhubei City (TW); Wen-Hsi WANG, Zhubei City (TW); Chua-Zu HUANG, Zhubei City (TW)

(73) Assignee: Taiwan Biomaterial Co., Ltd., Zhubei City, Hsinchu County (TW)

(21) Appl. No.: 17/615,134

(22) PCT Filed: Aug. 7, 2020
(86) PCT No.: PCT/CN2020/107891

§ 371 Date: Nov. 30, 2021

Related U.S. Application Data
(60) Provisional application No. 62/883,741, filed on Aug. 7, 2019.

(51) Int. Cl. A61B 17/22

(52) U.S. Cl. CPC A61B 17/22; A61B 2017/22079; A61B 2017/22051; A61B 2017/22038

(57) ABSTRACT

A thrombus removal device (100) comprising a catheter (10), at least one valve (20), and a shaft (30) is provided. The catheter (10) has a proximal end (12) and a distal end (11), and defines a longitudinal axis. The at least one valve (20) is positioned and movable longitudinally inside the catheter (10). The shaft (30) is connected with the proximal end (12) of the catheter (10), and comprises a driving mechanism and a wire (34). Wherein, the wire (34) is connected to the driving mechanism at one end and connected to the at least one valve (20) at an opposite end. The driving mechanism is configured to create a sudden movement of the at least one valve (20) toward the proximal end (12) of the catheter (10). By using the thrombus removal device (100), thrombus can be removed from the body of a subject more efficiently.

### CROSS-REFERENCE TO RELATED APPLICATIONS

The present disclosure claims priority to U.S. Provisional Application Ser. No. 62/883,741 filed on Aug. 7, 2019, the entirety of which is hereby incorporated by reference herein for all purposes.

### FIELD OF THE INVENTION

The present disclosure relates to a thrombus removal device, which is used to efficiently remove thrombus from the body of a subject.

### BACKGROUND OF THE INVENTION

Thrombosis is caused by abnormal coagulation of blood or debris that blocks the flow of blood in the vessel, which then causes hypoxia of adjacent tissues. Thrombosis is the main reason of ischemic stroke. Treatment of thrombosis focuses on removal of thrombus. Main stream treatment relies on thrombolytic medicines; however, the efficacy could vary from patient to patient. Interventional treatment is another option to remove thrombus in situ. Briefly, traditional interventional treatment introduces a device into the blood vessel to remove the thrombus mechanically. However, from time to time, the device might break the thrombus into pieces and larger pieces might cause thrombosis in other places of the blood vessel.

Another interventional treatment is related to the usage of negative pressure suction with a catheter. In a nutshell, the proximal end of the catheter is connected with a suction pump (or a syringe), and the distal end of the catheter is directed into a blood vessel. When the distal end of the catheter arrives the position of thrombus, the pump is used to suck the thrombus into the catheter by the production of the negative pressure so as to remove the thrombus from the patient's body. However, if the position of thrombus inside the patient's body is far away from the pump, the long catheter (i.e. long distance) will decrease the negative pressure created by the pump. Thus, the resulted negative pressure would be insufficient to remove the thrombus. Usually, the larger the inner diameter of the catheter, the stronger the pressure conserved. However, the larger catheter cannot be introduced into a small blood vessel and thus could be useless in many critical positions such as brain blood vessels. Another way to increase the negative pressure is to raise the suction power. However, as the maximum negative pressure created by the suction pump or syringe is 760 mmHg, it is limited to adjust or increase the pressure to over 760 mmHg.

In light of the foregoing, the field continuously needs a better solution to remove thrombus from the patient's body.

### SUMMARY OF THE INVENTION

In order to achieve the aforesaid objective, the present disclosure provides a thrombus removal device in which a negative pressure is produced in a position close to thrombus to be removed in a blood vessel.

In an aspect of the present disclosure, a thrombus removal device comprising a catheter, at least one valve, and a shaft is provided. The catheter has a proximal end and a distal end, and defines a longitudinal axis. The at least one valve is positioned and movable longitudinally inside the catheter. The shaft is connected with the proximal end of the catheter, and comprises a driving mechanism and a wire. Wherein, the wire is connected to the driving mechanism at one end and connected to the at least one valve at an opposite end. The driving mechanism is configured to create a sudden movement of the at least one valve toward the proximal end of the catheter.

Preferably, the driving mechanism comprises a supporting rod and a spring disposed around the supporting rod.

Preferably, the shaft further comprises a body, and the spring and a part of the supporting rod are disposed in the body.

Preferably, one end of the spring connects to the supporting rod and an opposite end of the spring connects to the body of the shaft.

Preferably, the driving mechanism further comprises a releasing member for holding or releasing the supporting rod.

Preferably, the driving mechanism further comprises a chunk connected to the wire.

Preferably, the shaft further comprises an O-ring for sealing a space in the body.

Preferably, the shaft is connected with the catheter through a connector.

Preferably, the thrombus removal device comprises a plurality of the valves connected to each other in series and disposed in the catheter.

Preferably, the valves are connected to each other through the wire.

Preferably, the at least one valve is a ball or an extendable structure.

Preferably, the extendable structure is a balloon type or an umbrella type.

Preferably, the elasticity coefficient of the spring is 50-1000 N/m.

In the present disclosure, there are at least the following advantages:

1. In contrast to negative pressure created in the proximal end of the catheter far away from the thrombus to be removed in the prior art, the negative pressure of the present disclosure is created in the distal end of the catheter near the thrombus.

2. The effect of the thrombus removal device in the present disclosure will not be influenced by the length of the catheter, and/or the location of thrombus.

3. The thrombus removal device of the present disclosure can provide stable negative pressure so as to safely remove thrombus from the individual.

### BRIEF DESCRIPTION OF THE DRAWINGS

FIG. 1 illustrates the operational schematic diagrams of a thrombus removal device to remove thrombus from a blood vessel according to an embodiment of the present disclosure.

FIG. 2 illustrates a plurality of valves connected to each other in series according to an embodiment of the present disclosure.

FIG. 3 illustrates the schematic diagram of a shaft connected with the catheter according to an embodiment of the present disclosure.

FIG. 4 illustrates the schematic diagram of a chunk connected to the wire of a shaft according to an embodiment of the present disclosure.

FIG. 5 illustrates the schematic diagram of a shaft without/with an O-ring according to an embodiment of the present disclosure.

FIG. 6 illustrates the schematic diagram of a shaft connected with a catheter through a connector according to an embodiment of the present disclosure.

FIG. 7 illustrates the schematic diagrams of a valve located at the distal end of a catheter according to an embodiment of the present disclosure.

FIG. 8 illustrates the operational schematic diagrams of a thrombus removal device according to an embodiment of the present disclosure.

FIG. 9 illustrates the operational schematic diagrams of a valve to remove thrombus from a blood vessel according to an embodiment of the present disclosure.

FIG. 10 illustrates the schematic diagrams of a thrombus removal device of (a) a connector, (b) a pump, and (c) a syringe according to an embodiment of the present disclosure.

FIG. 11 illustrates the operational schematic diagrams of a valve of single-ball type or multi-ball type to remove thrombus according to an embodiment of the present disclosure.

FIG. 12 illustrates the operational schematic diagrams of a valve of single-balloon type or multi-balloon type to remove thrombus according to an embodiment of the present disclosure.

FIG. 13 illustrates the operational schematic diagrams of a valve with single-umbrella type or multi-umbrella type to remove thrombus according to an embodiment of the present disclosure.

### DETAILED DESCRIPTION OF THE INVENTION

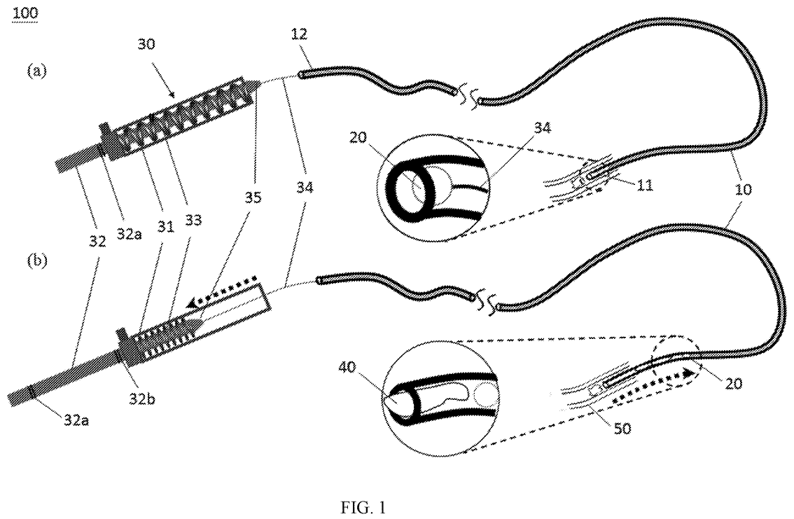

Please refer to the following FIG. 1, which illustrates a working example of the thrombus removal device of the present disclosure. As shown in FIG. 1, the thrombus removal device 100 comprises a catheter 10, a valve 20, and a shaft 30

The catheter 10 comprises a distal end 11 and a proximal end 12. The distal end 11 is to be introduced into a blood vessel 50 and eventually be positioned close to a thrombus to be removed (see part (a) of FIG. 1). The proximal end 12, on the other hand, is more likely to be positioned outside the body of the subject in need of thrombus removing. The proximal end 12 is connected to the shaft 30. The operator, such as a medical practitioner, can control the movement of the catheter 10 through the shaft 30.

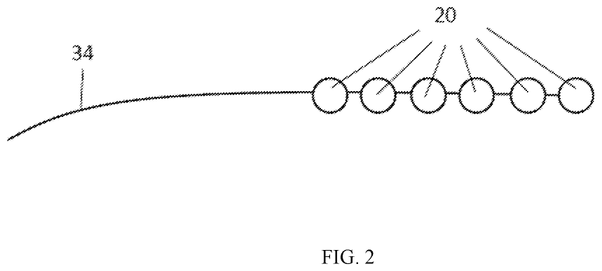

The valve 20 can be any shape while in the working example of FIG. 1, the valve 20 is spherical. In an embodiment, the valve 20 has a shape corresponding to the inner wall of the catheter 10. For example, the valve 20 is a sphere and has a diameter slightly shorter than the inner diameter of the catheter 10 so that the valve 20 is movable longitudinally inside the catheter 10 and is able to create a negative pressure while moving. In another embodiment, the thrombus removal device 100 comprises a plurality of valves 20. For example, the plurality of valves 20 are linked with each other in series through a wire 34 axially, as shown in FIG. 2. In other specific embodiments, the valve 20 can be an extendable structure, such as a balloon type or an umbrella type. These embodiments are particularly favorable for multiple action in one operation. Further elaboration will be provided in following paragraphs.

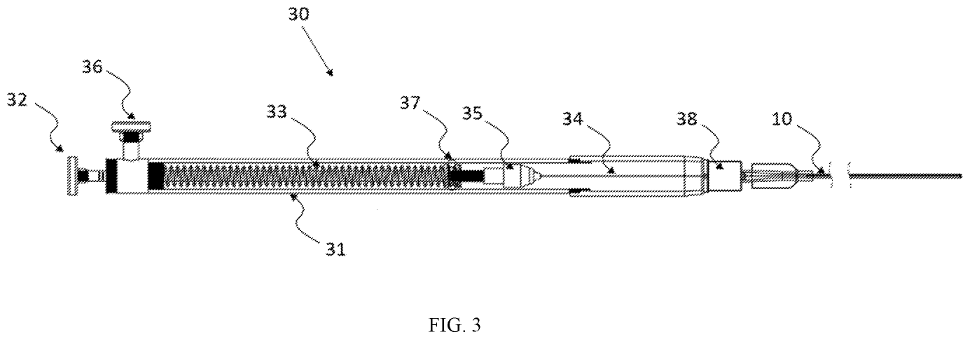

Please refer to FIGS. 1 and 3. The shaft 30 comprises a body 31, a supporting rod 32, a spring 33, and the wire 34. The spring 33 has one end connected to the supporting rod 32 and the other end connected to the body 31. In an embodiment, the elasticity coefficient of the spring 33 is 50-1000 N/m, preferably 100-600 N/m. Preferably, there are one or two indication lines 32a and/or 32b labeled on the supporting rod 32 to indicate a loaded position and/or a released position. The travel distance of the supporting rod 32 may range 2-20 cm, preferably 5-15 cm, wherein the travel distances means a distance from the released position to the loaded position. The end of the supporting rod 32 to be pressed may be formed of an elastic material, such as silicone or rubber, to reduce the impact as it is released from the loaded position. The body 31 is constructed for an operator to hold and has a releasing member 36. The supporting rod 32, the spring 33 and the releasing member 36 forms a driving mechanism for the valve 20. The releasing member 36 holds the supporting rod 32 in position and releases the same while being pushed. The releasing member 36 may be formed of an elastic material, such as silicone or rubber, to reduce the vibration while triggering.

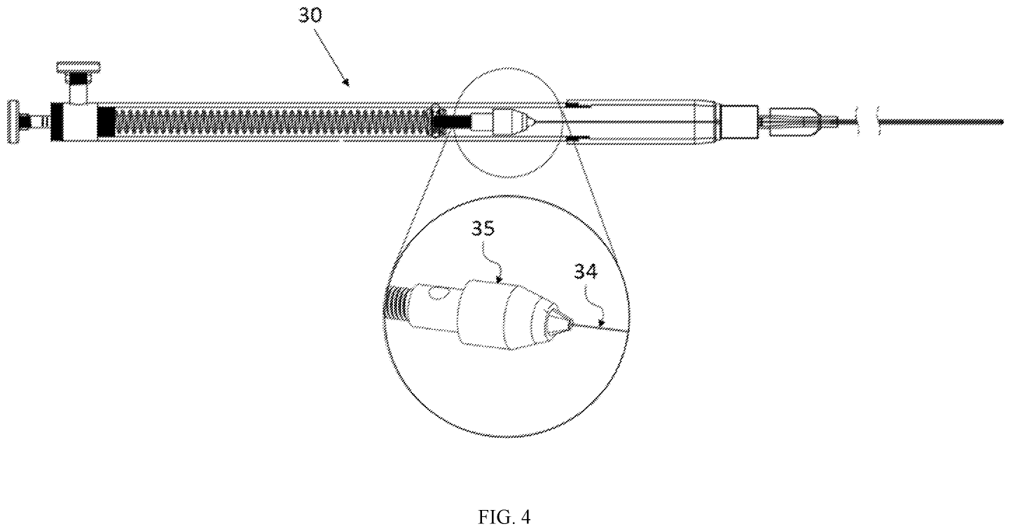

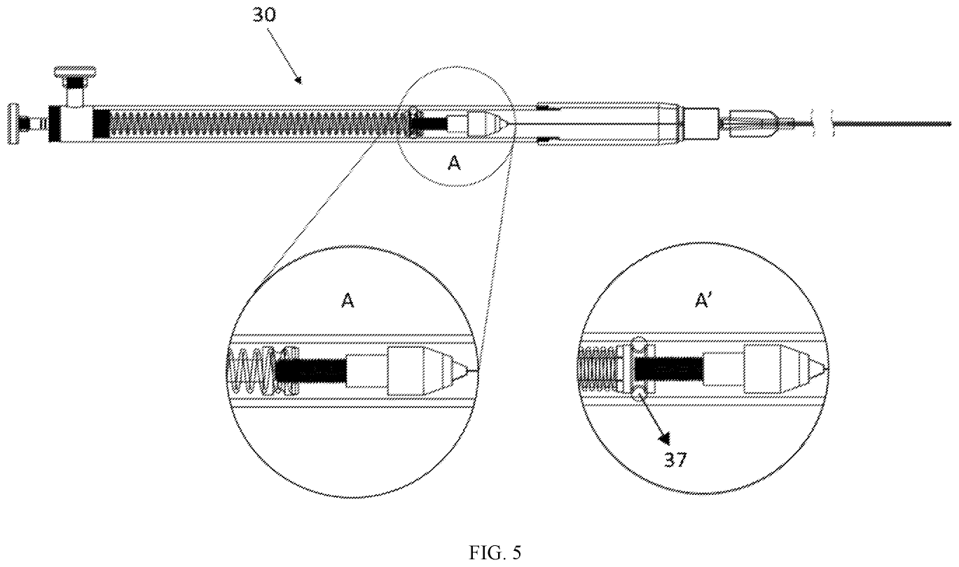

In an embodiment, the supporting rod 32 has a chuck 35 used to connect to the wire 34. For example, FIG. 4 shows the chuck 35 is connected to the wire 34 by clipping the wire 34. The wire 34 and the chuck 35 can be any biomedical compatible materials, such as stainless. Refer to FIG. 5, it shows embodiments of the shaft 30 with or without the O-ring 37. In an embodiment A′, the shaft 30 further comprises an O-ring 37 for sealing a space in the body 31 such that the negative pressure can be created and maintained more efficiently to increase suction force while the valve 20 is moving back to the proximal end 12 of the catheter 10. The O-ring 37 also enhances the stabilization of the movement of the supporting rod 32 inside the body 31.

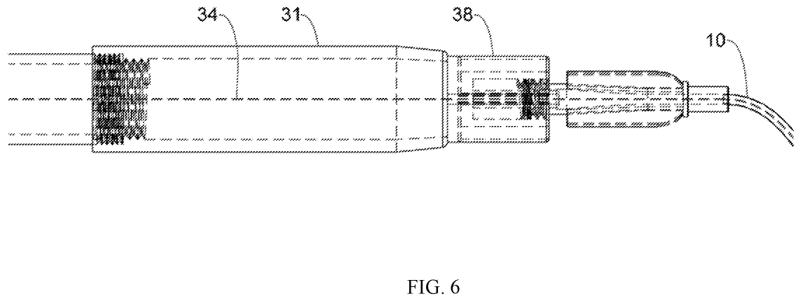

Please refer to FIG. 6. The shaft 30 further comprises a connector 38, which connects the catheter 10 with the body 31. In this embodiment, the connector 38 is set in the front of the shaft 30 and accommodates the wire 34 to pass therethrough.

Operation of Disclosed Thrombus Removal Device

After the position of the thrombus of a patient is verified, a medical practitioner can remove the thrombus by using the thrombus removal device of the present disclosure. In a preferable embodiment, the thrombus removal device of the present disclosure can be used simultaneously with an imaging technology such as an X-ray and/or an ultrasound device.

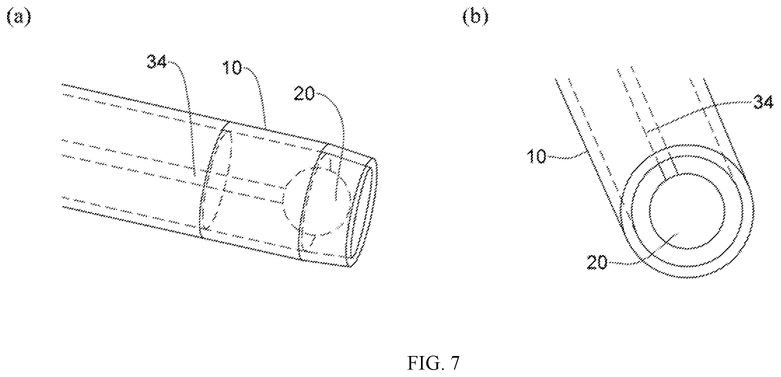

Please refer back to FIG. 1. The medical practitioner can hold and operate the disclosed thrombus removal device 100 through the shaft 30. First of all, a leading wire (i.e. guide wire; not shown in the figure) is introduced into the blood vessel 50 until the vicinity of the thrombus 40. Then, the catheter 10 is extended alongside the leading wire until the distal end 11 of the catheter 10 adjacent to the thrombus 40. The leading wire is drawn out after the catheter 10 is on position and the valve 20 is moved thereafter. The valve 20 is eventually positioned at the distal end 11 of the catheter 10 and basically at the edge thereof, as shown in FIG. 7. In other words, while the distal end 11 of the catheter 10 is adjacent to the thrombus 40 in the blood vessel 50, the valve 20 is at the position of the catheter 10 closest to the thrombus 40.

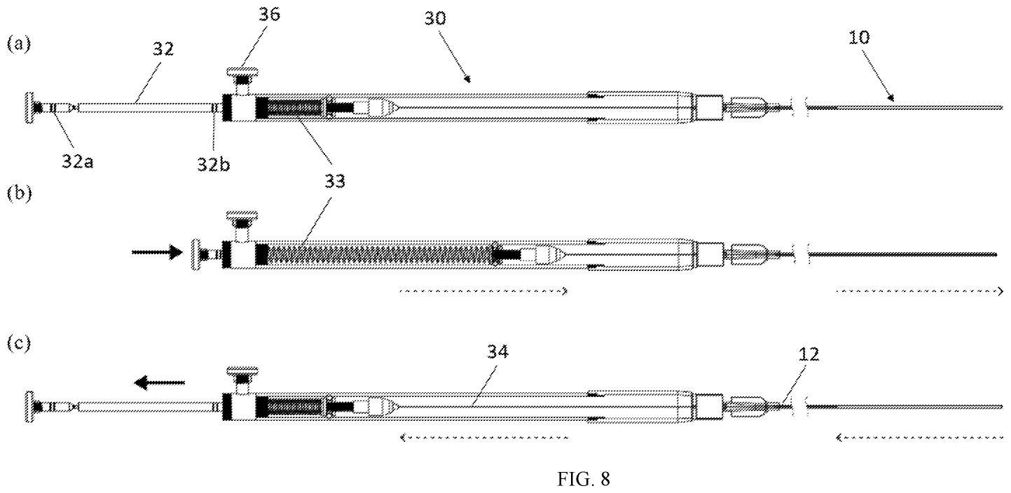

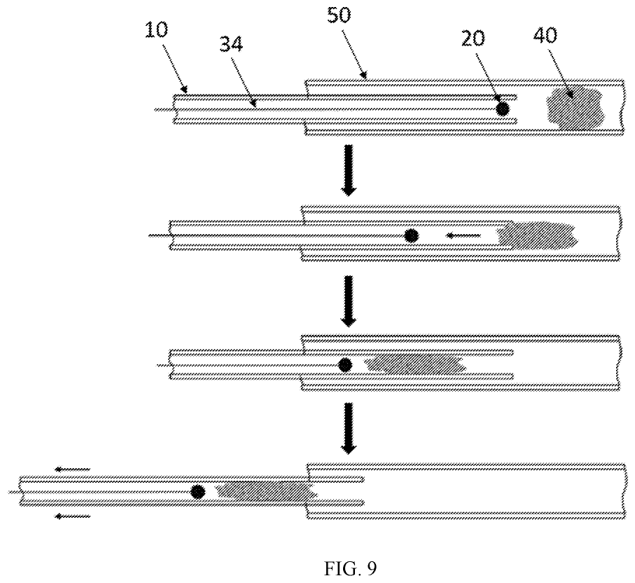

Then, please see part (b) of FIG. 1. After the catheter 10 and the valve 20 are at the desired position adjacent to thrombus 40 to be removed, the medical practitioner can push the releasing member 36 so as to release the position of the supporting rod 32. The released supporting rod 32 would be pushed backward by the spring 33. Consequently, a sudden movement of the valve 20 toward the proximal end 12 of the catheter 10 is initiated. In detail, please refer to FIG. 8 and FIG. 9 together. As shown in parts (a) and (b) of FIG. 8, the supporting rod 32 of the driving mechanism is pressed into a loaded status. Further refer to part (c) of FIG. 8. When the releasing member 36 is pressed, the supporting rod 32 will be released from the loaded status and quickly moved backward by the spring 33. While the supporting rod 32 is moving backward, the wire 34 connected to the driving mechanism will be also pulled backward so that the valve 20 moves towards the proximal end 12 of the catheter 10. As a result, at this moment, the pressure at the distal end 11 of the catheter 10 close to the thrombus 40 will be lower than the pressure at the vicinity of the thrombus 40 in the blood vessel 50. That is, the negative pressure is produced at the distal end 11 of the catheter 10 so as to push or draw the thrombus 40 into the catheter 10 (see FIG. 9).

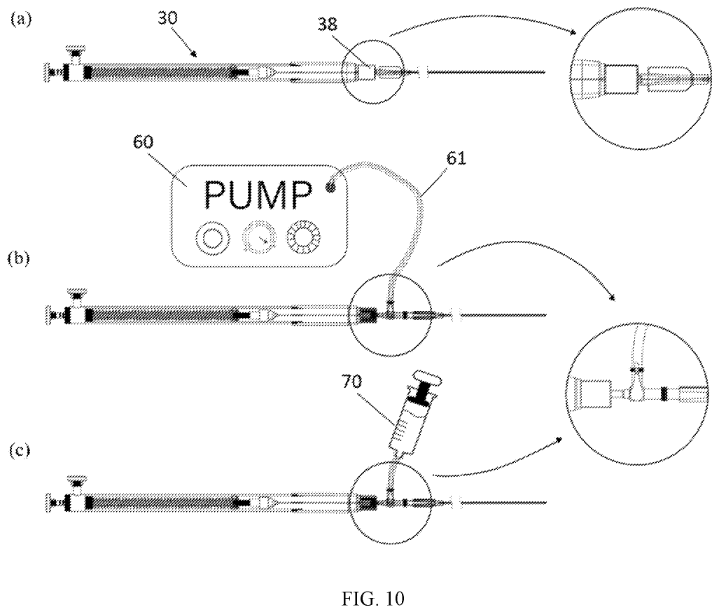

Briefly, without being bound by any theory, the sudden movement of the valve 20 would create a negative pressure at the adjacent position of the thrombus 40, thereby sucking the thrombus into the catheter 10. The thrombus 40 will then be moved from the blood vessel 50, transferred along the catheter 10, and finally arrived outside of the patient's body. In an embodiment, a part or all of the connector 38 of the shaft 30 can be exchanged to connect with an external pump 60 or syringe 70, as shown in parts (a) to (c) of FIG. 10. By this way, the thrombus 40 inside the catheter 10 could be easily and quickly took away from the catheter 10 by applying the pump 60 or syringe 70 with/without a tubing 61. In another embodiment, as the thrombus 40 has been sucked into the catheter 10, the medical practitioner can simply remove the catheter 10 together with the thrombus 40 so that the purpose of removing thrombus is met.

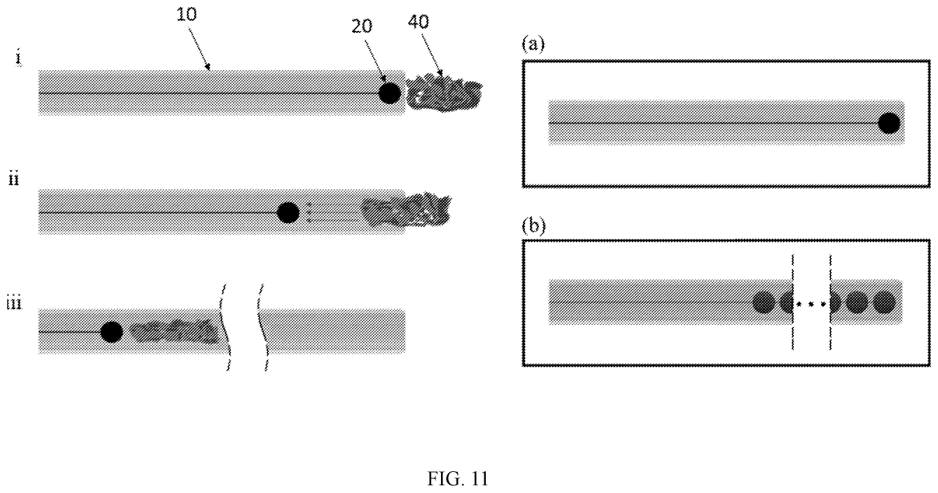

FIG. 11 illustrates the operational schematic diagrams of a valve of single-ball type or multi-ball type to remove thrombus according to an embodiment of the present disclosure. It further shows the corresponding movement of the ball-type valve 20 and the thrombus 40. If the thrombus cannot be removed once, the medical practitioner can reset the supporting rod 32, the wire 34, the ball-type valve 20, and the releasing member 36 back to an initial position and repeat the aforesaid action again without removing the catheter 10 from the patient's body.

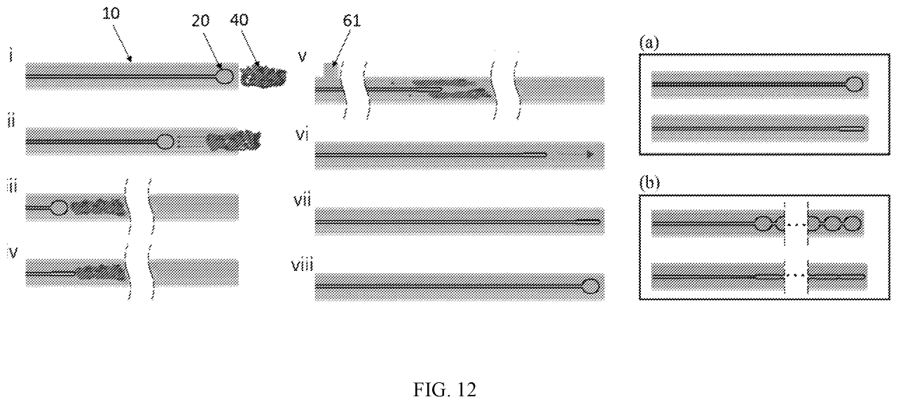

FIG. 12 illustrates the operational schematic diagrams of a valve of single-balloon type or multi-balloon type to remove thrombus according to an embodiment of the present disclosure. In FIG. 12, the catheter 10 and the balloon-type valve 20 are positioned adjacent to the thrombus 40 in the blood vessel (Action i). Then, a sudden movement of the balloon-type valve 20 toward the proximal end 12 of the catheter 10 is initiated (Action ii). The sudden movement of the balloon-type valve 20 would create a negative pressure at the adjacent position of the thrombus 40, thereby resulting in sucking the thrombus 40 into the catheter 10 (Actions ii and iii). In case that thrombus 40 is not fully removed or there is another thrombus needed to be removed, the balloon-type valve 20 can be shrunk to let the thrombus 40 pass (Action iv and v). Then, the shrunk valve 20 can be moved toward the next thrombus spot (Actions vi and vii) and inflated to repeat the aforesaid suction action again (Actions viii).

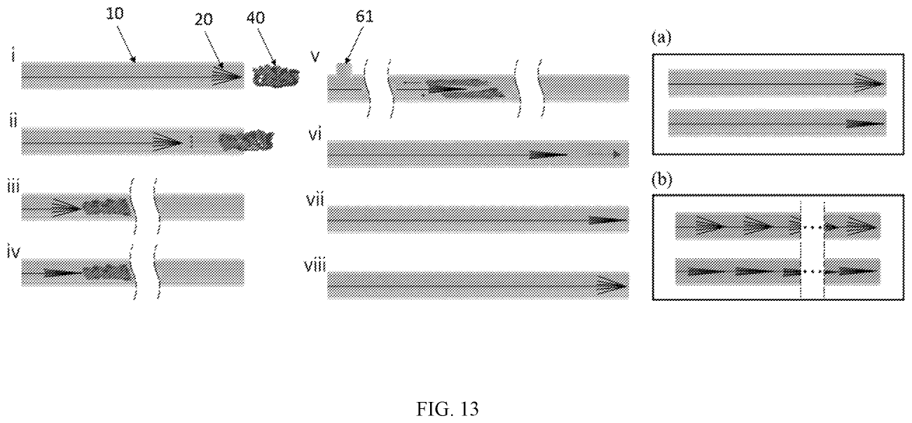

FIG. 13 illustrates the operational schematic diagrams of a valve of single-umbrella type or multi-umbrella type to remove thrombus according to an embodiment of the present disclosure. Likewise, the catheter 10 and the umbrella-type valve 20 are positioned adjacent to the thrombus 40 in the blood vessel (Action i). Then, the umbrella-type valve 20 is suddenly and quickly moved back to the proximal end 12 of the catheter 10 so as to produce a negative pressure at the distal end 11 of the catheter 10 adjacent to the thrombus 40, causing the thrombus being sucked into the catheter 10 (Actions ii and iii). The umbrella-type valve 20 can be folded and extended repeatedly so that multiple actions of continuously removing thrombus 40 can be achieved in one operation (Actions iv to viii). In light of the foregoing, the thrombus removal device of the present disclosure is convenient to operate and provides better efficiency for removing thrombus. It also can be equipped for the conventional and clinically-used thrombus removal devices to improve its efficiency.

What is claimed is:

1. A thrombus removal device, comprising:
a catheter having a proximal end and a distal end, and defining a longitudinal axis;
at least one valve positioned and movable longitudinally inside the catheter; and
a shaft connected with the proximal end of the catheter, and comprising a driving mechanism and a wire;
wherein the wire is connected to the driving mechanism at one end and connected to the at least one valve at an opposite end; wherein the driving mechanism is configured to create a sudden movement of the at least one valve toward the proximal end of the catheter.

2. The thrombus removal device of claim 1, wherein the driving mechanism comprises a supporting rod and a spring disposed around the supporting rod.

3. The thrombus removal device of claim 2, wherein the shaft further comprises a body, and the spring and a part of the supporting rod are disposed in the body.

4. The thrombus removal device of claim 3, wherein one end of the spring connects to the supporting rod and an opposite end of the spring connects to the body of the shaft.

5. The thrombus removal device of claim 2, wherein the driving mechanism further comprises a releasing member for holding or releasing the supporting rod.

6. The thrombus removal device of claim 1, wherein the driving mechanism further comprises a chunk connected to the wire.

7. The thrombus removal device of claim 1, wherein the shaft further comprises an O-ring for sealing a space in the body.

8. The thrombus removal device of claim 1, wherein the shaft is connected with the catheter through a connector.

9. The thrombus removal device of claim 1, comprising a plurality of the valves connected to each other in series and disposed in the catheter.

10. The thrombus removal device of claim 9, wherein the valves are connected to each other through the wire.

11. The thrombus removal device of claim 1, wherein the at least one valve is a ball or an extendable structure.

12. The thrombus removal device of claim 11, wherein the extendable structure is a balloon type or an umbrella type.

13. The thrombus removal device of claim 1, wherein an elasticity coefficient of the spring is 50-1000 N/m.
